(19) United States
(12) Patent Application Publication
(10) Pub. No.: US 2004/0096875 A1
(43) Pub. Date: May 20, 2004

(54) Novel gene for controlling leaf shapes

(76) Inventors: Hirohiko Hirochika, Tsukuba (JP); Akio Miyao, Toride (JP); Kazumasa Murata, Toyama (JP)

Correspondence Address: Richard M. Klein, Esq., Fay, Sharpe, Fagan, Minnich & McKee LLP, 7th Floor, 1100 Superior Avenue, Cleveland, OH, 44114, US

(21) Appl. No.: 10/619,685

(22) Filed: Jul. 15, 2003

Related U.S. Application Data
(62) Division of application No. 09/667,475, filed on Sep. 22, 2000, now abandoned.

(30) Foreign Application Priority Data
Mar. 23, 2000 (JP) 2000-83067

(51) Int. Cl. C12Q 1/68; C07H 21/04

(52) U.S. Cl. 435/6; 536/23.6

(57) ABSTRACT

There is provided a polynucleotide encoding a plant gene capable of controlling leaf shapes, the polynucleotide encoding an amino acid sequence from Met at position 1 to Val at position 690 of SEQ ID NO: 2 in the SEQUENCE LISTING, including any polynucleotide encoding an amino acid sequence in which one or more amino acids are deleted, substituted or added to the amino acid sequence.

### BACKGROUND OF THE INVENTION

1. Field of the Invention

The present invention relates to a novel gene. In particular, the present invention relates to a novel gene in plants which encodes a protein having the function of controlling leaf shapes.

2. Description of the Related Art

Transposons are mutagenic genes which are known to be ubiquitous in animal, yeast, bacterial, and plant genomes. Transposons are classified into two classes, Class I and Class II, depending on their transposition mechanisms. Transposons belonging to Class II are transposed in the form of DNAs without being replicated. Known Class II transposons include the Ac/Ds, Spm/dSpm and MU elements of Zea mays (Fedoroff, 1989, Cell 56, 181-191; Fedoroff et al., 1983, Cell 35, 235-242; Schiefelbein et al., 1985 Proc. Natl. Acad. Sci. USA 82, 4783-4787), and the Tam element of Antirrhinum majus (Bonas et al., 1984, EMBO J., 3, 1015-1019). Class II transposons are widely used for gene isolation techniques which utilize transposon tagging. Such techniques utilize the fact that a transposon induces physiological and morphological changes when inserted into genes. The affected gene can be isolated by detecting such changes (Bancroft et al., 1993, The Plant Cell, 5, 631-638; Colasanti et al., 1998, Cell, 93, 593-603: Gray et al., 1997, Cell, 89, 25-31; Keddie et al., 1998, The Plant Cell, 10, 877-887; Whitham et al., 1994, Cell, 78, 1101-1115).

Transposons belonging to Class I, also referred to as retrotransposons, are replicated and transposed via RNA intermediates. Class I transposons were first identified and characterized in Drosophila and in yeasts. However, recent studies have revealed that Class I transposons are ubiquitous in plant genomes and account for a substantial portion of the genomes (Bennetzen, 1996. Trends Microbiolo., 4, 347-353; Voytas, 1996, Science, 274, 737-738). A large majority of retrotransposons appear to be inactive. Recent studies indicate that some of these retrotransposons are activated under stress conditions such as injuries, pathogenic attacks, or cell culture (Grandbastien, 1998, Trends in Plant Science, 3, 181-187; Wessler, 1996, Curr. Biol. 6, 959-961; Wessler et al., 1995, Curr. Opin. Genet. Devel. 5, 814-821). Activation under stress conditions has been reported for Tnt1A and Tto1 in tobacco (Pouteau et al., 1994, Plant J., 5, 535-542; Takeda et al., 1988, Plant Mol. Biol., 36, 365-376), and Tos17 in rice (Hirochika et al., 1996, Proc. Natl. Acad. Sci. USA, 93, 7783-7788), for example.

The Tos17 retrotransposon of rice is one of the most-extensively studied plant Class I elements In plants. Tos17 was cloned by an RT-PCR method using a degenerate primer prepared based on a conservative amino acid sequence in reverse transcription enzyme domains between Ty1-copia retroelements (Hirochika et al., 1992, Mol. Gen. Genet., 233, 209-216). Tos17 is 4.3 kb long, and has two 138 bp LTRs (long chain terminal repetitions) and PBS (primer binding sites) complementary to the 3′ end of the start methionine tRNA (Hirochika et al., 1996, supra). Tos17 transcription is strongly activated through tissue culture, and its copy number increases with culture time. In Nipponbare, a model Japonica cultivar used for genome analysis, two copies of Tos17 are initially present, which are increased to 5 to 30 copies in a regenerated plant after tissue culture (Hirochika et al., 1996, supra). Unlike Class II transposons which were characterized in yeasts and Drosophila, Tos17 is transposed in chromosomes in random manners and causes stable mutation, and therefore provides a powerful tool for functional analysis of rice genes (Hirochika, 1997, Plant Mol. Biol. 35, 231-240: 1999, Molecular Biology of Rice (ed. by K. Shimamoto, Springer-Verlag, 43-58).

### SUMMARY OF THE INVENTION

The present invention relates to a polynucleotide encoding a plant gene capable of controlling leaf shapes, the polynucleotide encoding an amino acid sequence from M t at position 1 to Val at position 690 of SEQ ID NO: 2 in the SEQUENCE LISTING, including any polynucleotide encoding an amino acid sequence In which one or more amino acids are deleted, substituted or added to the amino acid sequence.

In one embodiment of the invention, the polynucleotide may be derived from rice.

In another embodiment of the invention, the polynucleotide may be an represented by SEQ ID NO: 1 in the SEQUENCE LISTING.

The present invention further relates to methods for controlling leaf shapes in plants.

The inventors diligently conducted systematic analyses of phenotypes of plants having a newly transposed To17 copy and sequences adjoining Tos17 target sites with respect to rice. As a result, the inventors found a narrow-leaf rice mutation obtained from Tos17 insertion, and isolated the gene responsible for this mutation by utilizing Tos17 as a tag, thereby accomplishing the present invention.

Thus, the invention described herein makes possible the advantage of: providing a novel plant gene which can be provided by using Tos17.

This and other advantages of the present invention will become apparent to those skilled in the art upon reading and understanding the following detailed description with reference to the accompanying figures.

### BRIEF DESCRIPTION OF THE DRAWINGS

FIG. 1 is a photograph showing a Tos17-inserted narrow-leaf mutant rice plant (left) and a wild-type rice plant (right).

FIG. 2 shows a Southern analysis autoradiogram of DNA extracted from self-crossed progeny from a narrow-leaf mutant NC0608 strain (R2 generation) and DNA extracted from a wild-type rice. On the left is shown a autoradiogram of a Southern analysis performed by using Tos17 as a probe. On the right is shown an autoradiogram of a Southern analysis performed by subcloning NC0608_{—}0_{—}102, which is one of the adjoining sequences of Tos17, and using it as a probe. The lane indicated as M is a lane of a λ/HindIII marker. The lane indicated as C is a control lane in which DNA obtained from a wild-type plant (Nipponbare) was electrophoresed. The lane indicated as mt is a lane in which DNA obtained from a narrow-leaf mutant was electrophoresed.

FIG. 3 is a schematic representation of a gene which control leaf shapes. Blank boxes in the figure represent introns, whereas black boxes represent exons. The downward arrow on the right-hand side of the figure represent a position at which Tos17 was inserted. The two small downward arrows near the 5′ end and the 3′ end represent a start codon site and a stop codon site, respectively.

### DESCRIPTION OF THE PREFERRED EMBODIMENTS

The present invention provides a novel plant gene which can be provided by using Tos17, a vector containing the same, a plant which is transformed by the novel gene, and a method of producing an improved plant including a stop of transforming a plant with the novel gene.

According to the present invention, there is provided a polynucleotide encoding a plant gene capable of controlling leaf shapes. As used herein, the term “Controlling leaf shapes” means the ability to alter the leaf length and/or leaf width of a plant, thereby enhancing photosynthesis ability or imparting resistance against lodging, etc. The term “plants” encompasses both monocotyledons and dicotyledons.

A polynucleotide encoding a plant gene capable of controlling leaf shapes according to the present invention is, for example, a polynucleotide encoding an amino acid sequence from Met at position 1 to Val at position 690 of SEQ ID NO: 2 in the SEQUENCE LISTING, including any polynucleotide encoding an amino acid sequence in which one or more amino acids are deleted, substituted or added to the aforementioned amino acid sequence.

A polynucleotide encoding a plant gene capable of controlling leaf shapes encompasses any polynucleotides which have at least about 80% sequence homology, preferably at least about 85% sequence homology, and more preferably at least about 90% sequence homology, still more preferably at least about 95% sequence homology, and most preferably at least about 99% sequence homology, with an amino acid sequence from Met at position 1 to Val at position 690 of SEQ ID NO: 2 in the SEQUENCE LISTING, so long as they are capable of controlling leaf shapes in plants. The term “sequence homology” indicates a degree of identicalness between two polynucleotide sequences to be compared with each other. The rate (%) of sequence homology between two polynucleotide sequences for comparison is calculated by, after optimally aligning the two polynucleotide sequences for comparison, obtaining a matched position number indicating the number of positions at which identical, or “matched”, nucleic acid bases (e.g., A, T, C, G, U, or I) are present in both sequences, dividing the matched position number by total number of bases in the polynucleotide sequences for comparison, and multiplying the quotient by 100. The sequence homology can be calculated by using the following sequencing tools, for example: a Unix base program designated GCG Wisconsin Package (Program Manual for the Wisconsin Package, Version 8, September. 1994, Genetics Computer Group, 575 Science Drive Madison, Wis., USA 53711; Rice, P. (1996) Program Manual for EGCG Package, Peter Rice, The Sanger Centre, Hinxton Hall, Cambridge, CB10 1RQ, England), and the ExPASy World Wide Web molecular biology server (Geneva University Hospital and University of Geneva, Geneva, Switzerland).

The term “control sequence” as used herein refers to a DNA sequence including a functional promoter and any related transcription elements (e.g., an enhancer, CCAAT box, TATA box, SPI site, etc.).

The term “operably linked” as used herein refers to a manner of linking a polynucleotide such that various regulation elements such as a promoter, enhancer, etc., which regulate its expression can operate within a host cell.

It in well-known to those skilled in the art that the type and kinds of control sequences may vary depending on the host cell. For example, CaMV35S promoter, nopaline synthase promoter, and the like are well-known to those skilled in the art. Any methods that are known to those skilled in the art may be used for introducing the gene into a plant body. For example, methods which utilize agrobacterium and methods which directly introduce a gene in a cell are well known. As for methods which utilize agrobacterium, the method of Nagel et al. (Microbiol. Lett. 67, 325 (1990)) may be used, for example. This method involves first transforming agrobacterium with an expression vector via electroporation, and then introducing the transformed agrobacterium into a plant cell by following a method described in Plant Molecular Biology Manual (S. B. Gelvin et al., Academic Press Publishers). Electroporation techniques and partile gun techniques are known as methods for directly introducing a gene into a cell.

Cells into which genes have been introduced are first selected based on drug resistance, e.g., hygromycin resistance, and then regenerated into plant bodies by using usual methods.

The terminology and laboratory procedures described throughout the present specification are directed to those which are well-known and commonly employed in the art. Standard techniques may be used for recombination methods, polynucleotide synthesis, microorganisms culturing, and transformation (e.g., electroporation). Such techniques and procedures are generally known from various standard textbooks available in the field or by way of the present specification (including a generally-referenced textbook by Sambrook at al., Molecular Cloning: A Laboratory Manual, 2nd edition (1989) Cold Spring Harbor Laboratory Press, Cold Spring Harbor, N.Y.). Such literature is incorporated herein by reference.

The polynucleotide according to the present invention can be obtained by using the method described herein, for example. However, the polynucleotide according to the present invention may also be obtained by any chemical synthesis process based on the sequence disclosed her in. For example, the polynucleotide according to the present invention may be synthesized by using a polynucleotide synthesizer available from Applied Bio Systems in accordance with the instructions provided by the manufacturer.

Methods of PCR amplification are well-known in the art (PCR Technology: Principles and Applications for DNA Amplification, ed. H A Erlich, Freeman Press, NewYork, N.Y. (1992); PCR Protocols: A Guide to Methods and Applications, Innis, Gelfland, Snisky, and White, Academic Press, San Diego, Calif.(1990); Mattila et al. (1991) Nucleic Acids REs. 19: 4967: Eckert, K. A. and Kunkel, T. A. (1991) PCR Methods and Applicatdions 1: 17 ;PCR, McPherson, Quirkes, and Taylor, IRL Press, Oxford). Such literature is incorporated herein by reference.

### EXAMPLES

Hereinafter, the present invention will be described by way of examples which are of illustrative but not limitative nature.

### Example 1

### Activation of Tos17 via Culture

Using fully ripened seeds of Nipponbare, which is a variety of Japonica subspecies, induction of calli and cell suspension culture were carried out as described earlier (Hirochika et al., 1996, supra). The activation of Tos17 was carried out following the method of Ohtsuki (1990) (rice protoplast culture system, Food and Agricultural Research Development Association). In summary, fully ripened seeds of rice were cultured in an MS medium having 2,4-dichlorophenoxyacetic acid (2,4-D) added thereto (2 mg/ml) (Ohtsuki (1990), supra) (25° C., 1 month), to induce callus formation. The resultant calluses were cultured for 5 months in an N6 liquid medium having 2,4-D added thereto (Ohtsuki (1990), supra), and thereafter placed on a redifferentiation medium (Ohtsuki (1990), supra), whereby redifferentiated rice plants were obtained (first generation (R1) plants).

### Example 2

### Isolation and Identification of Narrow-Leaf Mutants

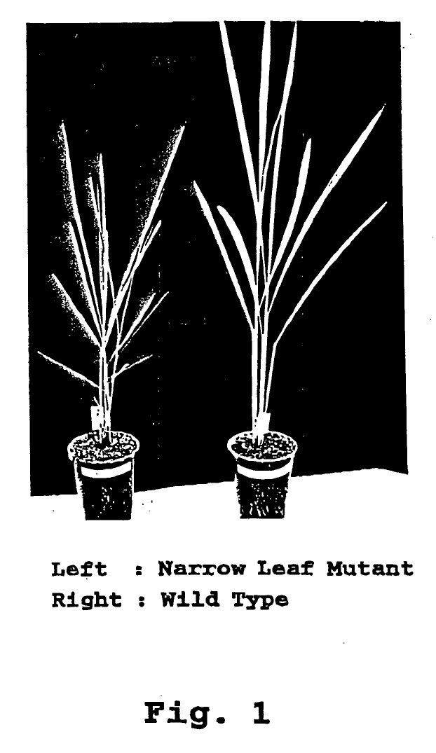

Utilizing each of the regenerated R1 rice plants obtained according to Example 1. about 1000 R1 seeds were collected from each strain and sown on a paddy field to obtain second generation (R2) plants, which were subjected to a morphological analysis. As a result of observing the phenotypes of the respective plant bodies in the R2 group, it was learned that about ¼ of the R2 group of the NC0608 strain exhibit the “narrow-leaf” phenotype (FIG. 1). In the paddy field, the Tos17-inserted narrow-leaf mutants had their leaf length reduced to about 90% in the flag leaf and all leaves down to the third leaf therefrom: and they also had their leaf width reduced to about 78%, about 70%, about 71%, about 69%, respectively, in the flag leaf and all leaves down to the third leaf therefrom (FIG. 1, left), as compared with the wild type (FIG. 1, right). This suggested that the narrow-leaf phenotype of NC06089 is caused by recessive mutation at a single gene locus.

### Example 3

### Isolation of Causative Gene for Narrow-Leaf Mutations

In order to identify and isolate the causative gene for narrow-leaf mutations from the NC0608 strain obtained according to Example 2, linkage analysis with respect to the Tos17 gene was performed on a group part of which was separable as narrow-leaf mutations. In order to show that recessive mutation at a single gene locus is responsible for the mutations, adjoining portions of a target site (Ts) of the NC0608 strain at which Tos17 had been transpos—inserted were amplified first.

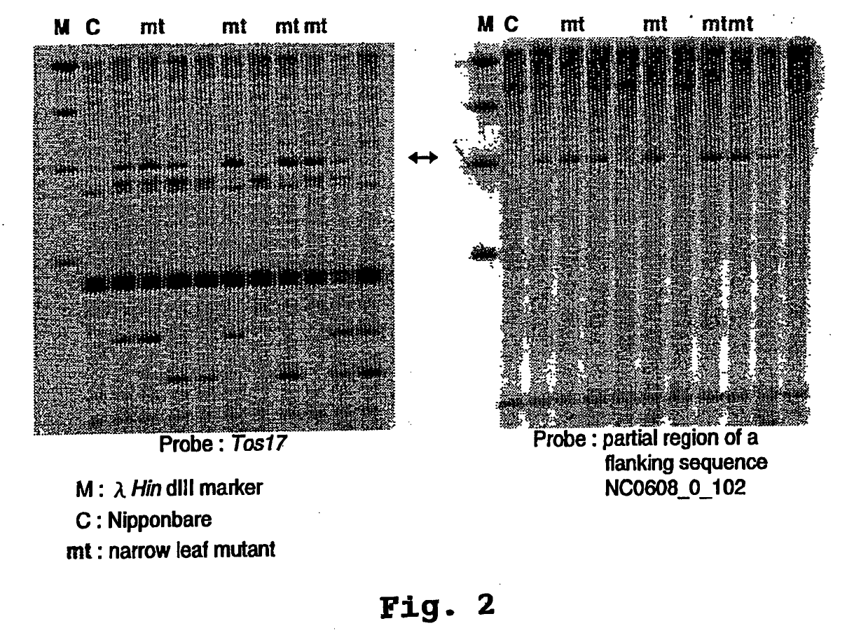

From the group of R2 rice plants (self-crossed progeny from the NC0608 strain) obtained according to Example 2, individuals exhibiting mutation were identified from normal individuals. DNA was prepared from both groups of individuals by using a CTAB method (Murray and Thompson, 1980, Nucleic Acids Res. 8, 4321-4325). The DNA obtained from individuals exhibiting narrow-leaf mutation and the DNA obtained from normal individuals were each digested with restriction enzyme XbaI, and after agarose electrophoresis, were allowed to adsorb to nylon membranes. DNA fragments which were obtained from Tos17 through digestion by XbaI and BamHI were labeled with ³²P-dCTP. By using these as probes, a Southern hybridization was performed (FIG. 2, left). As seen from the Southern analysts autoradiogram shown on the left-hand side in FIG. 2, it was learned the Tos17 band (about 6600 bp) indicated by an arrow was observed in narrow-leaf mutations as a homozygous band, but not in normal individuals, and that the Tos17 band indicated by the arrow was completely linked with the narrow-leaf mutation phenotype. From these results, It was concluded that the DNA which is represented by the band which hybridizes to the Tos17 probe indicated by the arrow contains a causative gene, such that Tos17, when inserted in a genome region represented by this band, generates narrow-leaf mutations as the genotype becomes homozygous. Accordingly, a portion of the causative gene for the narrow-leaf mutations, i.e., a sequence adjoining Tos17, was isolated through TAIL-PCR reactions using this DNA as a template. The amplification of the Tos17 target site sequence was accomplished by TAIL-PCR employing the total DNA (Liu Y -G. et al., 1995, Genomios, 25, 674-681, Liu Y -G. et al., 1995, Plant J., 8, 457-463). In summary, by using as a template the total DNA from a regenerated plant having a new Tos17 target site, three TAIL-PCR amplification reactions were performed, using the following three sets of primers: (1st reaction) Tos17 Tail3, GAGAGCATCATCGGTTACATCTTCTC and AD1 (arbitrarily degenerated primer 1) NGTCGA (G/C) (A/T) GANA (A/T) GAA; (2nd reaction) Tos17 Tail4, ATCCACCTTGAGTTTGAAGGG and AD1; and (3rd reaction) Tos17 Tail5, CATCGGATGTCCAGTCCATTG and AD1. Next, the respective TAIL-PCR products were subjected to an agarose electrophoresis and then a simple column purification. By directly applying them to a sequencer (Model 377 available from ABI), sequencing was performed.

Four new target sites (Ts) for Tos17 insertion were identified as a result of sequencing the adjoining sequences of Tos17 In the NC0608 strain.

Next, a Southern analysts was performed by subcloning NC0608_{—}0_{—}102, one of the adjoining sequences of Tos17, and using it as a probe. The results are shown on the right-hand side in FIG. 2. As seen from the autoradiogram on the right-hand side in FIG. 2, the Tos17-adjoining sequence NC0608_{—}0_{—}102 hybridized to the DNA fragment located at the same position as that indicated in the Southern analysis in which Tos17 was used as a probe. The results were consistent for all of the 62 strains that were examined. This indicates that the subclone NC0608_{—}0_{—}102 contains a portion of the causative gene for the narrow-leaf mutation, and that NC0608_{—}0_{—}102 is an adjoining sequence of the causative gene for the narrow-leaf mutation.

### Example 4

### Structural Analysis of the Causative Gene for Narrow-Leaf Mutation

Relying on the adjoining sequence obtained according to Example 3, the inventors attempted to determine the complete structure of cDNA which was transcribed from the gene containing the adjoining sequence NC0608_{—}0_{—}102 through a PCR screening using a cDNA library and Cap Site cDNA (Nippongene). By using the wild-type (Nipponbare) DNA as a template, the inventors attempted to determine the complete structure of the genomic DNA of the gene containing NC0608_{—}0_{—}102 through a PCR using a primer which is designed from the cDNA and through the aforementioned TAIL-PCR.

The cDNA library was previously prepared in the laboratory of the inventors. The method of preparation can be summarized as follows. First, by using an ISOGEN solution (Nippongene), the total RNA was extracted from a callus of a wild-type rice plant which had been cultured in the aforementioned MS medium. By using an oligo(dT)cellulose column contained in an mRNA purification kit (Stratagen), poly(A)mRNA was obtained from the total RNA. Following usual methods, cDNA was synthesized from the resultant poly(A)mRNA. Thus, a cDNA library was constructed in a Hybri ZAP-II vector (Stratagene).

The cDNA and genomic DNA of the gene containing the adjoining sequence NC0608_{—}0_{—}102 were partially amplified through the below-described four-step PCR reactions and three-step PCR reactions, respectively. All of the amplified fragments were sequenced by using a 377 sequencer (Perkin Elmer) for both directions.

### cDNA

First step: Using cDNA library as a template, a PCR reaction was carried out by using a pair of primers specific to the adjoining sequence NC0608_{—}0_{—}102 to confirm that a portion of this adjoining sequence is contained in the cDNA library: NC0608_{—}0_{—}102F ACGGAGACACCTCGTAAACC and NC0608_{—}0_{—}102R1 AAGGCCGACTATTGTTGACC.

Second step: Using the cDNA library as a template, a PCR reaction was carried out by using NC0608_{—}0_{—}102F and Hybri ZAP B (Stratagene), which is a primer specific to Hybri ZAP-II vector. Thus, a fragment which partially overlaps with NC0608_{—}0_{—}102 and which contains the 3′ region of cDNA along with the poly(A) binding site was obtained.

Third step: Using the cDNA library as a template, a PCR reaction was carried out by using Hybri ZAP A (Stratagene), which is a primer specific to Hybri ZAP-II vector, and NC0608_{—}0_{—}102R2 CCTGCAATGTTACCTCTGGC, which is a primer specific to NC0608_{—}0_{—}102. Thus, a 5′ fragment which partially overlaps with NC0608_{—}0_{—}102 was obtained.

Fourth step: Using Cap Site cDNA (Nipponegne) as a template, a PCR reaction was carried out by using 1RC2 (Nippongene), which is a primer specific to Cap Site, and TGACAGGTCAGACTGATCAACCGG, which is a primer specific to the fragment obtained in the third step. Thus, a fragment which partially overlaps with the fragment obtained in the third step and which contains the 5′ region of cDNA along with the transcription start point (cap site).

### Genomic DNA

First step: Using the total DNA of Nipponbare, two reactions of TAIL-PCR were carried out using the following two sets of primers to obtain a 5′ fragment which partially overlaps with the NC0608_{—}0_{—}102: (first reaction: NC0608_{—}0_{—}102R2 and AD1 employed in Example 3; second reaction: NC0608_{—}0_{—}102R3 TAGGCAATCCGGCAATGTCC and AD1)

Second step: Using the total DNA of Nipponbare, a PCR reaction was carried out using a primer (CTAGAAGCAAAATCTTGAAGCTGC) which is specific to the fragment obtained in the first step and a primer (AGTGTTCTTCGCACCTCGCG) which is specific to the cDNA fragment obtained in the fourth step PCR. Thus, a 5′ fragment which partially overlaps with the fragment obtained in the first step was obtained.

Third step: Using the total DNA of Nipponbare, a PCR reaction was carried out using a primer (TGCCTCGCCCTCGGCGATGG) which is specific to the fragment obtained in the second step and a primer (AATATTTCAAATCACACTAC) which is specific to the 5′ region of the cDNA fragment obtained in the fourth step PCR. Thus, a 5′ fragment which partially overlaps with the fragment obtained in the second step was obtained.

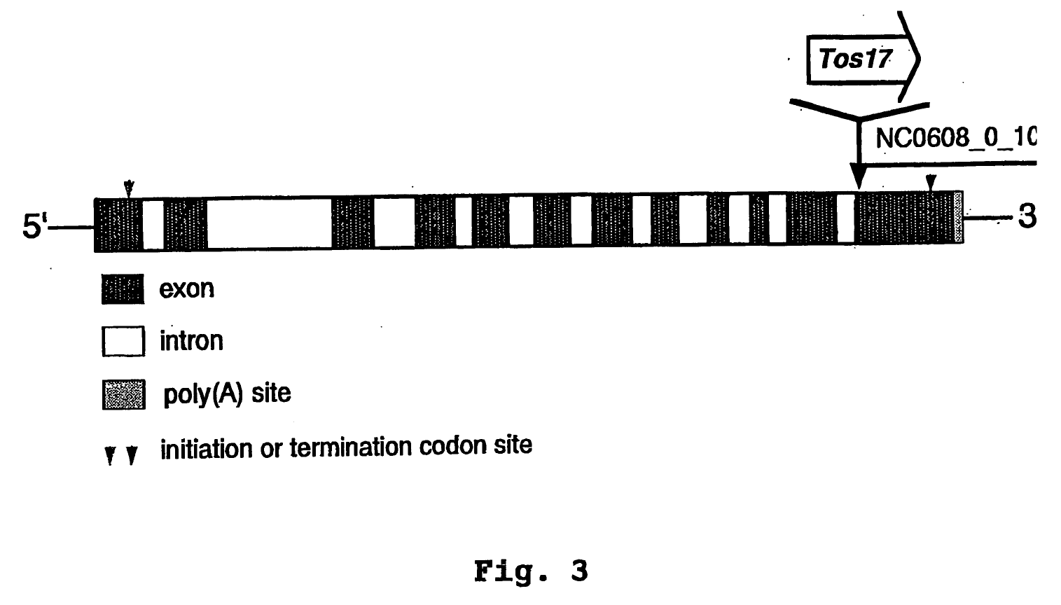

The cDNA and genomic DNA structures of the narrow-leaf gene are shown together in FIG. 3. This gene has 11 introns and encodes 690 amino acids, and yet finds no similar genes registered in existing databases. Thus, it was confirmed that this gene is novel. It was learned that Tos17 had been inserted between the 9th and the 10th bases from the 5′ end of the 12th exon region. An amino acid sequence encoded by this gene showed very high homology with a gene in Arabidopsis thaliana having an unknown function.

The above examples are illustrative, and by no means limitative, of various aspects of the present invention and the manners in which the oligonucleotide according to the present invention can be made and utilized.

Thus, according to the present invention, a novel polynucleotide is provided which in capable of controlling leaf shapes, the polynucleotide being of use in plant breeding. By introducing the present polynucleotide into plants and artificially controlling leaf shapes, it is expected that enhancement of photosynthesis ability or provision of resistance against lodging, etc., can be attained.

Various other modifications will be apparent to and can be readily made by those skilled in the art without departing from the scope and spirit of this invention. Accordingly, it is not intended that the scope of the claims appended hereto be limited to the description as set forth herein, but rather that the claims be broadly construed.

What is claimed is:

1. A polynucleotide encoding a plant gene capable of controlling leaf shapes, the polynucleotide encoding an amino acid sequence from Met at position 1 to Val at position 690 of SEQ ID NO: 2 in the SEQUENCE LISTING, including any polynucleotide encoding an amino acid sequence in which one or more amino acids are deleted, substituted or added to the amino acid sequence.

2. A polynucleotide according to claim 1 derived from rice.

3. A polynucleotide according to claim 1 as represented by SEQ ID NO: 1 in the SEQUENCE LISTING.


SEQUENCE LISTING (15 sequences)

SEQ ID NO: 1 - DNA, 2468 bp, Oryza sativa
  CDS (198)..(2270)
aaaaaaatatttcaaatcacactacactctccgtcgtctcctctcctctcctctcctccccctctcctccgcctctctcgcatctgaggctccgatcgccggcgaccccagccagaatccgccgccccgtctcgccctccccgctcgacgagaccgcgccgagcggcgaagaggcctagtgttcttcgcacctcgcgatgagtagcgcggtgaaggaccagcttcaccagatgtcgacgacatgcgattcgcttctactggagctcaatgtgatttgggatgaggtcggtgagcccgacacgacgagggacaggatgctgctggagctcgagcaggagtgcctggaggtctacaggcggaaggtcgaccaggcgaaccggagccgcgcccagctgcggaaggccatcgccgagggcgaggcagagctcgccggcatctgctcagccatgggcgagccgcccgtgcacgttagacagtcaaatcagaagcttcatggcttaagagaggagttgaatgcaattgttccgtatttggaagaaatgaaaaagaaaaaggtcgaacgatggaaccagtttgttcatgtcatagagcagattaagaaaatttcgtctgaaataaggccagccgattttgttccctttaaagttccggttgatcagtctgacctgtcattaagaaagcttgatgagttgacgaaggacctggaatcccttcagaaggagaagagcgatcggctaaagcaagtgatagaacatttgaattctttgcattccttatgtgaggtgcttggcatagatttcaagcaaacagtatatgaggtgcaccctagcttggacgaagctgaaggatcaaagaacctgagcaacactacaattgagaggcttgctgctgccgcaaacagactgcgtgaaatgaagatccaaaggatgcaaaagcttcaagattttgcttctagcatgctcgagctatggaatctcatggatactccacttgaagagcagcagatgtttcagaatataacatgcaatattgctgcttcagaacaagagataactgaaccaaacaccctctccacagatttcctgaattatgtcgaatctgaggtgttaaggcttgaacaactgaaagcaagtaagatgaaagatcttgttttaaaaaagaaagcagaactagaagagcatagaagacgtgctcatcttgttggcgaggaaggttatgcagaggagtttagcattgaagctattgaagctggagctattgatccctcactagtacttgaacaaattgaagctcacattgcaacagtgaaagaggaagcttttagccggaaggatattcttgagaaagttgaaagatggcaaaatgcttgtgaagaggaagcctggctggaagattacaacaaagatgataatcgttacaatgctgggaggggagcacatctaacactaaagagggctgaaaaggctcgtactttggtcaacaagattcctggaatggtagatgttttgagaacaaaaattgctgcatggaaaaatgaacgaggaaaggaggatttcacatatgatggtgttagcctttcgtcaatgcttgatgaatatatgttcgttcgtcaggagaaagagcaagagaagaagagacaaagggatcagaagaagctccaggatcagctcaaagcggagcaggaagctttgtacggatcaaaacccagtccatccaagcccctaagtacaaagaaggcacctaggcactctatgggtggtgcaaaccgaaggctatctcttggtggagccaccatgcaacccccgaagactgatatactgcattcaaagtctgttcgtgctgccaagaaaactgaagaaatcggcactttgtcccctagtagtagtagaggtttggacattgccggattgcctatcaagaagttgtctttcaatgccagtactctacgtgagacggagacacctcgtaaaccttttgctcagatcacaccaggaaacagtgtctcgtcgacgcctgtgcgccctatcaccaataacactgaggatgatgagaacaggactccgaagacatttacagcactgaatcccaagactccgatgactgttacggctccaatgcagatggcaatgactccctctctggccaacaaggtttcagcaactccagtttcccttgtttacgacaagccagaggtaacattgcaggaggacatcgactactcctttgaagaaaggcggctcgccatctatctggccaggcaaatggtttaactgttgatcaatttatgtacgtagttgaaatctgactgcattttcttgtcggtggccattgcgtatgttggtcaacaatagtcggcctttccagtagcactattctgatttactgcaattgttttaatgttttctacaaccagtaaaacagctctatacattagcttgctcactaaaaaaaaaaaaaaaaaaaaaaaa

SEQ ID NO: 2 - protein, 690 aa, Oryza sativa
MSSAVKDQLHQMSTTCDSLLLELNVIWDEVGEPDTTRDRMLLELEQECLEVYRRKVDQANRSRAQLRKAIAEGEAELAGICSAMGEPPVHVRQSNQKLHGLREELNAIVPYLEEMKKKKVERWNQFVHVIEQIKKISSEIRPADFVPFKVPVDQSDLSLRKLDELTKDLESLQKEKSDRLKQVIEHLNSLHSLCEVLGIDFKQTVYEVHPSLDEAEGSKNLSNTTIERLAAAANRLREMKIQRMQKLQDFASSMLELWNLMDTPLEEQQMFQNITCNIAASEQEITEPNTLSTDFLNYVESEVLRLEQLKASKMKDLVLKKKAELEEHRRRAHLVGEEGYAEEFSIEAIEAGAIDPSLVLEQIEAHIATVKEEAFSRKDILEKVERWQNACEEEAWLEDYNKDDNRYNAGRGAHLTLKRAEKARTLVNKIPGMVDVLRTKIAAWKNERGKEDFTYDGVSLSSMLDEYMFVRQEKEQEKKRQRDQKKLQDQLKAEQEALYGSKPSPSKPLSTKKAPRHSMGGANRRLSLGGATMQPPKTDILHSKSVRAAKKTEEIGTLSPSSSRGLDIAGLPIKKLSFNASTLRETETPRKPFAQITPGNSVSSTPVRPITNNTEDDENRTPKTFTALNPKTPMTVTAPMQMAMTPSLANKVSATPVSLVYDKPEVTLQEDIDYSFEERRLAIYLARQMV

SEQ ID NO: 3 - DNA, 4574 bp, Oryza sativa
aaaaaaatatttcaaatcacactacactctccgtcgtctcctctcctctcctctcctccccctctcctccgcctctctcgcatctgaggctccgatcgccggcgaccccagccagaatccgccgccccgtctcgccctccccgctcgacgagaccgcgccgagcggcgaagaggcctagtgttcttcgcacctcgcgatgagtagcgcggtgaaggaccagcttcaccagatgtcgacgacatgcgattcgcttctactggagctcaatgtatgtcaccgcttgccgattcaaccatttcccggctactcgtgttggttctggcatggcagtggaggatttacggggtttttttcttctctcgttctgtttcaggtgatttgggatgaggtcggtgagcccgacacgacgagggacaggatgctgctggagctcgagcaggagtgcctggaggtctacaggcggaaggtcgaccaggcgaaccggagccgcgcccagctgcggaaggccatcgccgagggcgaggcagagctcgccggcatctgctcagccatgggcgagccgcccgtgcacgttagacaggttagtttctggctccaccaatggctgtaaaagaggtatcgcatggttggatcaaaagatggaagtcgaattcctgtggaactgtgctaattggcgatggaagaaaaggaagatttagtagagaactaaaagctacgatttctgttgtaagatgatagtactactgcttgcattgttgatctgatggaggtaaaccgtgtagaactccatcagcagttaacatttttctaactgattagtagtagcgtatcaatatattaagggaaagtgttggcagagcttacatttctttctcacttctattctcgactttatgcccagttactgctcaatcggttctatactttttactgctgttcccatgcattagcaatttaggatatatgttttgtaaaatttatctgtttccttcagtttgaatatgttcagcatgaataatatatttactgttttaccggcagcatgactaagttactgcctcaagtacgttttatttgttgaatacattctaccttcttgactaatcaattctgcttgactgtagattttagcacttcctcagccattcatgcagtaacatgcatttcatctgaaattttgcagtcaaatcagaagcttcatggcttaagagaggagttgaatgcaattgttccgtatttggaagaaatgaaaaagaaaaaggtcgaacgatggaaccagtttgttcatgtcatagagcagattaagaaaatttcgtctgaaataaggccagccgattttgttccctttaaagttccggttgatcagtctgacctgtcattaagaaagcttgatgagttgacgaaggacctggaatcccttcagaaggagaaggtcatcatcactaataccatctttatccattttcaccagtcatgttgtcatcgtgtctctatctatcaagaatccttttcatttcttgtataaaatctcactatgccatatacatgtttgtttctcacagagcgatcggctaaagcaagtgatagaacatttgaattctttgcattccttatgtgaggtgcttggcatagatttcaagcaaacagtatatgaggtgcaccctagcttggacgaagctgaaggatcaaagaacctgagcaacactacaattgagaggcttgctgctgccgcaaacagactgcgtgaaatgaagatccaaaggatgcaaaaggtcagcattgcctgtaccattgtagaggtatcaatgaacactttcagtctttaacttggttaatctgattctggcagcttcaagattttgcttctagcatgctcgagctatggaatctcatggatactccacttgaagagcagcagatgtttcagaatataacatgcaatattgctgcttcagaacaagagataactgaaccaaacaccctctccacagatttcctgaattatgtaatttatcatcactgagattgcaaaaatttatgttcgtactgtgttatattttcattaagatatgaatgttcatcgactatacttataactgtaggtcgaatctgaggtgttaaggcttgaacaactgaaagcaagtaagatgaaagatcttgttttaaaaaagaaagcagaactagaagagcatagaagacgtgctcatcttgttggcgaggaaggttatgcagaggagtttagcattgaagctattgaagctggtaagatactctcctgccttactgccttttattgtgcctgacaagtcataccagacagagttcatatacctggtctgtgttctgttcgcaggagctattgatccctcactagtacttgaacaaattgaagctcacattgcaacagtgaaagaggaagcttttagccggaaggatattcttgagaaagttgaaagatggcaaaatgcttgtgaagaggaagcctggctggaagattacaacaaagtatggatgctagctgaagctacgtggtctttgtatatttgtttagcaaataatgtggtactgatatctcctggctttggctttttttaggatgataatcgttacaatgctgggaggggagcacatctaacactaaagagggctgaaaaggctcgtactttggtcaacaagattcctggtaatgttactcaatgatttatgtgtttggaacttccttatcaagtgcatatttaatttacaattttaactcttgccattactacaatctgatatcctgctgatttgtgctgagcaggaatggtagatgttttgagaacaaaaattgctgcatggaaaaatgaacgaggaaaggaggatttcacatatgatggtgtaggttttcttactcttacacattacattgatcgggtctatttttgtttcttgctgaagtgcctttcttgcaattcttacaggttagcctttcgtcaatgcttgatgaatatatgttcgttcgtcaggagaaagagcaagagaagaagagacaaagggtattatgctctcgcctaatattcatgtattgtctaaatcatcttttcaccttctgtgaatacgctctaatacttgaatatacctgcaggatcagaagaagctccaggatcagctcaaagcggagcaggaagctttgtacggatcaaaacccagtccatccaagcccctaagtacaaagaaggcacctaggcactctatgggtggtgcaaaccgaaggctatctcttggtggagccaccatgcaacccccgaagactgatatactgcattcaaagtctgttcgtgctgccaagaaaactgaagaaatcggcactttgtcccctagtaagccctactagctatcatgtgtcgatatatttctttttcctcttattttcacttgaacatatgtctaactcaagcaaacaatatcaggtagtagaggtttggacattgccggattgcctatcaagaagttgtctttcaatgccagtactctacgtgagacggagacacctcgtaaaccttttgctcagatcacaccaggaaacagtgtctcgtcgacgcctgtgcgccctatcaccaataacactgaggatgatgagaacaggactccgaagacatttacagcactgaatcccaagactccgatgactgttacggctccaatgcagatggcaatgactccctctctggccaacaaggtttcagcaactccagtttcccttgtttacgacaagccagaggtaacattgcaggaggacatcgactactcctttgaagaaaggcggctcgccatctatctggccaggcaaatggtttaactgttgatcaatttatgtacgtagttgaaatctgactgcattttcttgtcggtggccattgcgtatgttggtcaacaatagtcggcctttccagtagcactattctgatttactgcaattgttttaatgttttctacaaccagtaaaacagctctatacattagcttgctcactactcagtacagctttctcggcagcacgaaacatttctgttctctttgatgaatacttcttgctgtggatagggatagttactgttacatatactgtatgcccttcagaatagaaacctgttagtacgggaggtattataggaaggatcgttttggaattttggtggttagcctgcacagtaagttccatcagtttctggattgtccctcgcaaagaaaaaagttttcttgattctggtaattcgtttgtcccacctgactccttgaaagtcttctggacatgggaagctatcgtatcgtatcgctcgggcgaacatgatgtgtgtgtcactctcgagtgagcaggccaccgaaggctgacttgactgactccagcaaccaacaaacgagccagtcatttttcaccccgggtttttgtcccaaaacacttttccaccaccgtcaagcctcaagcaaaaccaaaacgctacgtaacgcccatcaacaccatgaaatcgagcagctagttgtgcctgctactggccccccagtgccctgtaccgcccgttcttctcactcgaca

SEQ ID NO: 4 - DNA, 26 bp, Artificial Sequence
  primer_bind: PCR primer
gagagcatcatcggttacatcttctc

SEQ ID NO: 5 - DNA, 21 bp, Artificial Sequence
  primer_bind: PCR primer
atccaccttgagtttgaaggg

SEQ ID NO: 6 - DNA, 21 bp, Artificial Sequence
  primer_bind: PCR primer
catcggatgtccagtccattg

SEQ ID NO: 7 - DNA, 20 bp, Artificial Sequence
  primer_bind: PCR primer
acggagacacctcgtaaacc

SEQ ID NO: 8 - DNA, 20 bp, Artificial Sequence
  primer_bind: PCR primer
aaggccgactattgttgacc

SEQ ID NO: 9 - DNA, 20 bp, Artificial Sequence
  primer_bind: PCR primer
cctgcaatgttacctctggc

SEQ ID NO: 10 - DNA, 24 bp, Artificial Sequence
  primer_bind: PCR primer
tgacaggtcagactgatcaaccgg

SEQ ID NO: 11 - DNA, 20 bp, Artificial Sequence
  primer_bind: PCR primer
taggcaatccggcaatgtcc

SEQ ID NO: 12 - DNA, 24 bp, Artificial Sequence
  primer_bind: PCR primer
ctagaagcaaaatcttgaagctgc

SEQ ID NO: 13 - DNA, 20 bp, Artificial Sequence
  primer_bind: PCR primer
agtgttcttcgcacctcgcg

SEQ ID NO: 14 - DNA, 20 bp, Artificial Sequence
  primer_bind: PCR primer
tgcctcgccctcggcgatgg

SEQ ID NO: 15 - DNA, 20 bp, Artificial Sequence
  primer_bind: PCR primer
aatatttcaaatcacactac